(19) United States
(12) Patent Application Publication
(10) Pub. No.: US 2022/0409124 A1
(43) Pub. Date: Dec. 29, 2022

(54) Method For Contactlessly Measuring Amount of Menstrual Blood In Menstrual Cup

(71) Applicant: GENIECUP INC., Ulsan (KR)

(72) Inventors: Yijin CHOI, Gyeonggi-do (KR); Taeyang YANG, Gyeonggi-do (KR)

(21) Appl. No.: 17/780,231

(22) PCT Filed: Oct. 15, 2020
(86) PCT No.: PCT/KR2020/014065

§ 371 Date: May 26, 2022

(30) Foreign Application Priority Data
Nov. 29, 2019 (KR) 10-2019-0157500

(51) Int. Cl. A61B 5/00; A61B 5/15; A61F 5/455; A61B 5/157; G01F 22/00; G01K 11/30

(52) U.S. Cl. CPC A61B 5/4325; A61B 5/150045; A61F 5/4553; A61B 5/157; G01F 22/00; G01K 11/30; A61B 2562/0204; A61B 2562/0271

(57) ABSTRACT

The present invention relates to a method for contactlessly measuring the amount of menstrual blood in a menstrual cup. The amount of menstrual blood that has been stored in a menstrual cup can be simply measured by using a contactless sensor means, thereby enabling the user to periodically check the amount of menstrual blood. This enables early detection and treatment of uterine fibroid, which may even lead to hysterectomy.

### TECHNICAL FIELD

The following description relates to a method of contactlessly measuring an amount of menstrual blood in a menstrual cup, and more particularly, to a contactless menstrual blood measurement method that easily measures an amount of menstrual blood stored in a menstrual cup using a contactless sensor means.

### BACKGROUND ART

For the prediction of the next menstrual date, women generally record a menstrual start date and count the 28th day from the menstrual start date to calculate the next menstrual day.

However, this method has low accuracy and the inconvenience of women having to manually record the calculated menstrual date.

On the other hand, since an increase in an amount of the menstrual blood may be a major indicator of the onset of uterine fibroid, it is required to measure an amount of menstrual blood every menstrual cycle. For the measurement of the amount of menstrual blood, the hospital uses an ineffective method of inserting a tool such as syringes into a woman's vagina and extracting the menstrual blood by time slot.

However, such a method may involve a complicated and inconvenient extraction process, and thus may not be readily applicable to the daily life of ordinary women.

Accordingly, there is a need for a method of measuring an amount of menstrual blood, which may be easily applied to daily life of ordinary women to simply measure an amount of menstrual blood and automatically record a menstrual cycle based on the measured amount of the menstrual blood.

### DISCLOSURE OF INVENTION

### Technical Goals

An aspect provides a contactless method of measuring an amount of menstrual blood in a menstrual cup that may simply measure an amount of menstrual blood stored in a menstrual cup using a contactless sensor means.

### Technical Solutions

According to an aspect, there is provided a method of contactlessly measuring an amount of menstrual blood in a menstrual cup, including the steps of: preparing a menstrual cup from which menstrual blood stored therein is emptied after the menstrual cup is removed from a female body; preparing a contactless sensor means including a contactless temperature sensor and a distance sensor which is coupled to one side of the contactless temperature sensor and has a measurement direction perpendicular to a measurement direction of the contactless temperature sensor; arranging an axial center of the menstrual cup on the same line with the measurement direction of the distance sensor, and measuring temperature values of specific points on an inner surface of the menstrual cup and distance values between the specific points on the inner surface of the menstrual cup and a lower end of the inner surface of the menstrual cup while moving the menstrual cup in a direction that the contactless sensor means is received in the menstrual cup; comparing the measured temperature values of the specific points on the inner surface of the menstrual cup, and specifying a distance value of a specific point on the inner surface of the menstrual cup at which a difference in temperature value occurs; and calculating an amount of menstrual blood by using the specified distance value of the specific point on the inner surface of the menstrual cup.

In this example embodiment, the contactless temperature sensor is an infrared sensor while the distance sensor is an ultrasonic sensor.

### Advantageous Effects

According to example embodiments described herein, an amount of menstrual blood that has been stored in a menstrual cup may be simply measured by using a contactless sensor means, enabling a user to periodically check an amount of menstrual blood, and thus detect and treat uterine fibroid early that may be even developed to fetal hysterectomy.

### BRIEF DESCRIPTION OF DRAWINGS

FIG. 1 is a flowchart illustrating a method of contactlessly measuring an amount of menstrual blood in a menstrual cup according to an example embodiment.

FIGS. 2A through 5 are diagrams for explaining a method of contactlessly measuring an amount of menstrual blood in a menstrual cup.

### BEST MODE FOR CARRYING OUT THE INVENTION

Hereinafter, the present disclosure is to be described in more detail with reference to the drawings. It should be noted that the same components in the drawings are denoted by the same reference numerals wherever possible. In addition, detailed descriptions of well-known functions and configurations that may unnecessarily obscure the gist of the present invention are omitted.

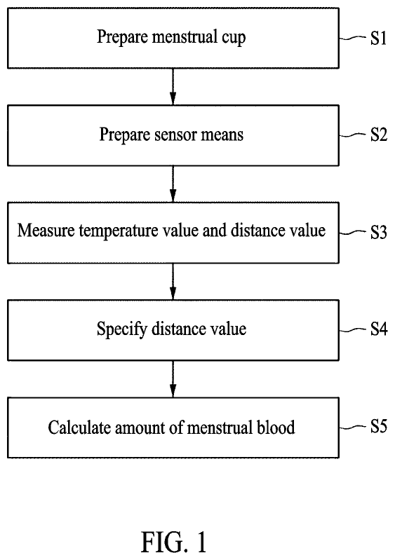

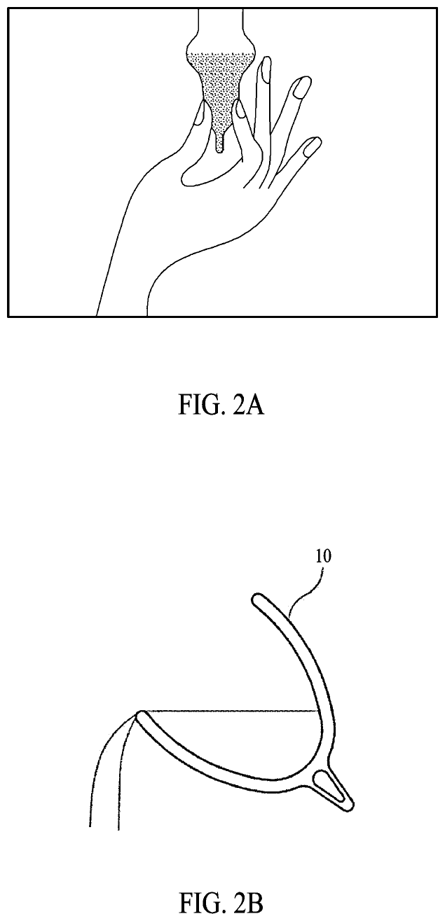

FIG. 1 is a flowchart illustrating a method of contactlessly measuring an amount of menstrual blood in a menstrual cup according to an example embodiment, and FIGS. 2A through 5 are diagrams for explaining a method of contactlessly measuring an amount of menstrual blood in a menstrual cup according to an example embodiment.

Referring to FIG. 1, the method for contactlessly measuring the menstrual blood in the menstrual cup according to an example embodiment may include step S1 of preparing a menstrual cup, step S2 of preparing a sensor means, step S3 of measuring temperature and distance values, step S4 of specifying distance values and step S5 of calculating an amount of menstrual blood.

Step S1 of preparing a menstrual cup may include removing a menstrual cup 10 storing menstrual blood from a female body as shown in FIG. 2A, emptying the menstrual blood stored in the menstrual cup 10 as shown in FIG. 2B, and preparing a menstrual cup from which the menstrual blood is removed as shown in FIG. 2C.

In this case, the menstrual cup from which the menstrual blood is removed, as shown in FIG. 2C, may have a difference in temperature between upper and lower parts that are open based on a portion of the menstrual cup filled with the menstrual blood in step S1.

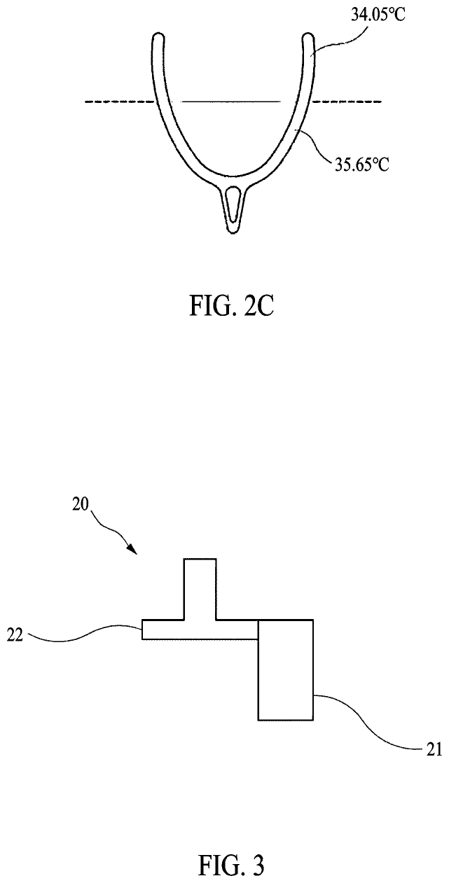

Step S2 of preparing a sensor means may include preparing a contactless sensor means 20 including a contactless temperature sensor 21 and a distance sensor 22 coupled to one side of the contactless temperature sensor 21 with its measurement direction being arranged perpendicularly to a measurement direction of the contactless temperature sensor 21, as shown in FIG. 3.

In this step, the contactless temperature sensor 21 may be an infrared temperature sensor configured to measure a temperature by sensing heat emitted from a measurement surface, and the distance sensor 22 may be an ultrasonic sensor which measures a distance by measuring a time used until receiving a reflected wave of an ultrasonic wave transmitted to the measurement surface.

It is obvious that the contactless temperature sensor 21 and the distance sensor 22 as described above are well-known enough to be used by those skilled in the art by purchasing commercially available ones in step S2.

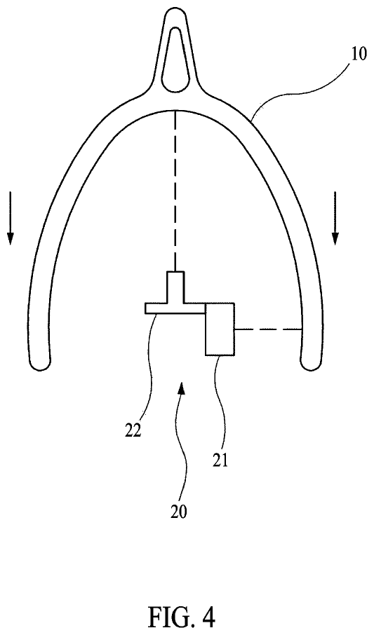

Step S3 of measuring temperature and distance values may include arranging an axial center of the menstrual cup to be on the same line with the measurement direction of the distance sensor 22, as shown in FIG. 4, and measuring temperature values of specific points on the inner surface of the menstrual cup 10 via the contactless temperature sensor 21 and measuring distance values between the specific points on the inner surface of the menstrual cup 10 and a center of a lower end of the inner surface of the menstrual cup 10 via the distance sensor 22 while moving the menstrual cup 10 in such a direction that the contactless sensor means 20 is contained in the menstrual cup 10 in step S3.

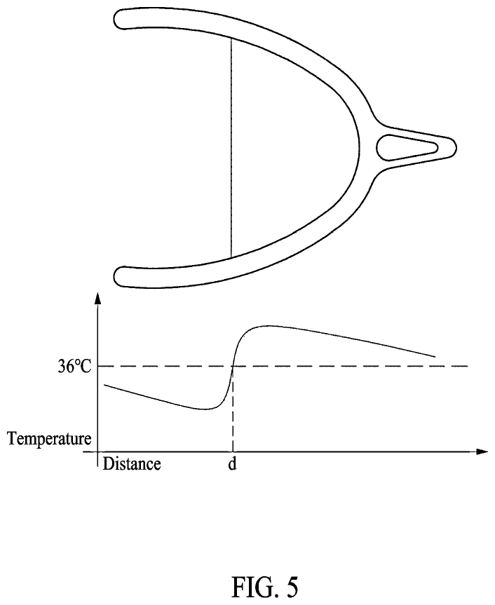

Step S4 of specifying distance values may include comparing the temperature values of the specific points on the inner surface of the menstrual cup based on a one-dimensional temperature distribution graph according to a distance between the specific points on the inner surface of the menstrual cup, as shown in FIG. 5, using the measured temperature values of the specific points on the inner surface of the menstrual cup and the measured distance values of the specific points on the inner surface of the menstrual cup, and specifying a distance value d of a specific point on the side of the menstrual cup at which a temperature difference occurs in step S4.

Step S5 of calculating an amount of menstrual blood may include calculating an amount of the menstrual blood having been stored in the menstrual cup, using the specified distance value d of the specific point on the inner surface of the menstrual cup.

The amount of menstrual blood may be a volume of an inner space of the menstrual cup containing the menstrual blood, which may be calculated in advance according to the distance values of the specific points of the menstrual cup in the form of a look-up table in step S5.

As described above, the method of contactlessly measuring an amount of menstrual blood in a menstrual cup according to an example embodiment may simply measure an amount of menstrual blood having been stored in the menstrual cup via the contactless sensor means, so that users may periodically check the amount of menstrual blood. This enables early detection and treatment of uterine fibroid, which may even lead to hysterectomy.

Although the present disclosure has been described with reference to the above preferred embodiments, various modifications and variations may be made without departing from the spirit and scope of the invention. Accordingly, the scope of the claims attached hereto is to cover such modifications and variations which fall within the subject matter of the present invention.

1.-3. (canceled)

4. A method of contactlessly measuring an amount of menstrual blood in a menstrual cup, comprising the steps of:
preparing a menstrual cup from which menstrual blood stored therein is emptied after the menstrual cup is removed from a female body;
receiving a contactless sensor including a contactless temperature sensor and a distance sensor which is coupled to the contactless temperature sensor and has a measurement direction perpendicular to a measurement direction of the contactless temperature sensor;
aligning the measurement direction of the distance sensor with a lower end of an inner surface of the menstrual cup, and measuring temperature values of specific points on the inner surface of the menstrual cup and distance values between the specific points on the inner surface of the menstrual cup and the lower end of the inner surface of the menstrual cup while moving the menstrual cup relative to the contactless sensor;
comparing the measured temperature values of the specific points on the inner surface of the menstrual cup, and specifying a distance value of a specific point on the inner surface of the menstrual cup at which a difference in temperature value occurs; and
calculating an amount of menstrual blood by using the specified distance value of the specific point on the inner surface of the menstrual cup.

5. The method of claim 4, wherein the contactless temperature sensor is an infrared sensor.

6. The method of claim 4, wherein the distance sensor is an ultrasonic sensor.
